(19) United States
(12) Patent Application Publication
(10) Pub. No.: US 2021/0298753 A1
(43) Pub. Date: Sep. 30, 2021

(54) ANASTOMOSIS DEVICE

(71) Applicant: SS INNOVATIONS CHINA CO., LTD., Hangzhou (CN)

(72) Inventors: Bryan D. Knodel, Flagstaff, AZ (US); Sudhir Prem Srivastava, Midland, TX (US); Daniel T. Wallace, Santa Cruz, CA (US)

(73) Assignee: SS INNOVATIONS CHINA CO., LTD., Hangzhou (CN)

(21) Appl. No.: 17/196,606

(22) Filed: Mar. 9, 2021

Related U.S. Application Data
(60) Provisional application No. 62/987,258, filed on Mar. 9, 2020.

(51) Int. Cl. A61B 17/115

(52) U.S. Cl. CPC A61B 17/115; A61B 2017/07257

(57) ABSTRACT

An Anastomosis Device is intended to create an end-to-side anastomosis between a graft vessel and a target vessel. The anastomosis is created by the delivery of staples that connect the graft vessel to the target vessel and the creation of an incision to open a flow path between the graft vessel and the target vessel.

### CROSS REFERENCE TO RELATED APPLICATION

The present application claims priority to and the benefit from U.S. Provisional Patent Application No. 62/987258 entitled “AN ANASTOMOSIS DEVICE” and filed Mar. 9, 2020, the contents of which are incorporated herein by reference in their entirety.

### FIELD OF THE DISCLOSURE

The present invention generally relates to a robotic surgical system for minimally invasive surgery. More particularly, the invention relates to an improved anastomosis device for the robotic surgical system.

### BACKGROUND OF THE DISCLOSURE

This section is intended to introduce the reader to various aspects of art that may be related to various aspects of the present disclosure, which are described below. This disclosure is believed to be helpful in providing the reader with background information to facilitate a better understanding of the various aspects of the present disclosure. Accordingly, it should be understood that these statements are to be read in this light, and not just as admissions of prior art.

Robotically assisted surgical systems have been adopted worldwide to replace conventional surgical procedures to reduce amount of extraneous tissue(s) that may be damaged during surgical or diagnostic procedures, thereby reducing patient recovery time, patient discomfort, prolonged hospital tenure, and particularly deleterious side effects. In robotically assisted surgeries, the surgeon typically operates a master controller at a surgeon console to seamlessly capture and transfer complex actions performed by the surgeon giving the perception that the surgeon is directly articulating surgical tools to perform the surgery. The surgeon operating on the surgeon console may be located at a distance from a surgical site or may be located within an operating theatre where the patient is being operated.

The robotically assisted surgeries have revolutionized the medical field and one of the fastest growing sectors in medical device industry. However, the major challenge in robotically assisted surgeries is to ensure the safety and precision during the surgery. One of the key areas of robotically assisted surgeries is the development of surgical robots for minimally invasive surgery. Over the last couple of decades, surgical robots have evolved exponentially and has been a major area of innovation in the medical device industry.

The robotically assisted surgical systems comprises of multiple robotic arms aiding in conducting robotic surgeries. The robotically assisted surgical system utilizes a sterile barrier to separate the non-sterile section of the robotic arm from a mandatory sterile surgical instrument attached to the robotic arm at an operating end. The sterile barrier may include a sterile plastic drape that envelops the robotic arm and a sterile adapter that operably engages with the sterile surgical instrument in a sterile field. The sterile barrier also may include a flexing drape interface to retain a drape section therebetween such that the torque and other force feedbacks is received as an input from both the sterile surgical instrument as well as the robotic arm. The sterile barrier is maintained between the sterile surgical instrument and the non-sterile robotic system. The sterile adapter detachably engages with an actuator assembly which drives and controls the sterile surgical instrument in a sterile field.

During the 1990s, there was an increasing number of surgeons using anastomosis devices such as surgical staples, rather than conventional sutures. This is true because the use of surgical staples and surgical stapling instruments has made many difficult procedures much simpler to perform. Of more importance, however, is that the use of surgical staples significantly reduces the time required for most procedures, and therefore reduces the length of time which the patient must be maintained under anesthetic. Many types of surgical stapling instruments have been devised for different surgical procedures.

In recent times, there is a rise of surgical stapler instruments where the surgical stapler is connected to a firing lever by an actuator cable where the firing lever actuates a staple driver to drive the staples into the tissue and against an anvil of the surgical stapler. During the use of these instruments, the force or load applied by to an operator to the actuator cable is non-uniform over the required firing stroke. The load is low during early portions of the stroke when the staples are advancing out of the cartridge and piercing the tissue. Once the staples bottom in an anvil pockets, the resistance and load rise rapidly as the staple legs buckle. Then the resistance and load drop down and rise again as the final forming of the staples into a B-shaped configuration occurs. In contrast, the operator has maximum effective strength at the mid-portion of the stroke of the instrument. At the final portion of the stroke, it is advantageous to require a lower operating force to make it easier to over-travel another lever for reopening of the instrument. In addition, it is easier for the operator to complete the firing stroke if the input load drops off at the end of the stroke.

In addition, there has been increasing number of adverse events associated with the surgical staplers in the past few years with some of the most commonly reported problems in these adverse event reports include: opening of the staple line or malformation of staples, misfiring, difficulty in firing, failure of the stapler to fire the staple and misapplied staples.

In the light of aforementioned challenges, there is a need for a robotic surgical system with an improved anastomosis device that allows ease, safety, and efficient use of staples for associated surgeries.

### BRIEF DESCRIPTION OF THE DRAWINGS

To further clarify advantages and features of the present disclosure, a more particular description of the disclosure will be rendered by reference to specific embodiments thereof, which is illustrated in the appended drawings. It is appreciated that these drawings depict only typical embodiments of the disclosure and are therefore not to be considered limiting of its scope. The disclosure will be described and explained with additional specificity and detail with the accompanying drawings in which:

FIG. 1(a) illustrates a schematic diagram of multiple robotic arms of a robotic surgical system in accordance with an embodiment of the disclosure;

FIG. 1(b) illustrates a schematic diagram of a surgeon console of the robotic surgical system in accordance with an embodiment of the disclosure;

FIG. 1(c) illustrates a schematic diagram of a vision cart of the robotic surgical system in accordance with an embodiment of the disclosure;

FIG. 2 illustrates a perspective view of a tool interface assembly mounted on a robotic arm in accordance with an embodiment of the disclosure;

FIG. 3(a) illustrates a perspective view of an anastomosis device in accordance with an embodiment of the disclosure;

FIG. 3(b) illustrates a perspective view of an end effector of the anastomosis device in accordance with an embodiment of the disclosure;

FIG. 3(c) illustrates a perspective view of a manual end effector of the anastomosis device in accordance with an embodiment of the disclosure;

FIG. 3(d) illustrates a perspective view of a robotic end effector of the anastomosis device in accordance with an embodiment of the disclosure;

FIG. 3(e) illustrates a perspective view of a cartridge portion of the anastomosis device in accordance with an embodiment of the disclosure;

FIG. 3(f) illustrates another perspective view of the cartridge portion of the anastomosis device in accordance with an embodiment of the disclosure;

FIG. 3(g) illustrates a perspective view of the components of cartridge portion of the anastomosis device in accordance with an embodiment of the disclosure;

FIG. 3(h) illustrates a perspective view of a wedge of cartridge of the anastomosis device in accordance with an embodiment of the disclosure;

FIG. 3(i) illustrates a perspective view of an anvil portion of end effector of the anastomosis device in accordance with an embodiment of the disclosure;

FIG. 3(j) illustrates a sectional view of the anvil portion of end effector of the anastomosis device in accordance with an embodiment of the disclosure; and

FIG. 3(k) illustrates a sectional view of a knife portion of end effector of the anastomosis device in accordance with an embodiment of the disclosure.

### DETAILED DESCRIPTION OF THE DISCLOSURE

For the purpose of promoting an understanding of the principles of the disclosure, reference will now be made to the embodiment illustrated in the drawings and specific language will be used to describe the same. It will nevertheless be understood that no limitation of the scope of the disclosure is thereby intended, such alterations and further modifications in the illustrated system, and such further applications of the principles of the disclosure as illustrated therein being contemplated as would normally occur to one skilled in the art to which the disclosure relates.

It will be understood by those skilled in the art that the foregoing general description and the following detailed description are exemplary and explanatory of the disclosure and are not intended to be restrictive thereof. Throughout the patent specification, a convention employed is that in the appended drawings, like numerals denote like components.

Reference throughout this specification to “an embodiment,” “another embodiment,” “an implementation,” “another implementation,” or similar language means that a particular feature, structure, or characteristic described in connection with the embodiment is included in at least one embodiment of the present disclosure. Thus, appearances of the phrase “in an embodiment,” “in another embodiment,” “in one implementation,” “in another implementation,” and similar language throughout this specification may, but do not necessarily, all refer to the same embodiment.

The terms “comprises,” “comprising,” or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures proceeded by “comprises . . . a” does not, without more constraints, preclude the existence of other devices or other sub-systems or other elements or other structures or additional devices or additional sub-systems or additional elements or additional structures.

Unless otherwise defined, all technical and scientific terms used herein have the same meaning as commonly understood by one of ordinary skill in the art to which this disclosure belongs. The apparatus, system, and examples provided herein are illustrative only and not intended to be limiting.

The terms “a” and “an” herein do not denote a limitation of quantity, but rather denote the presence of at least one of the referenced items. Further, the term sterile barrier and sterile adapter denotes the same meaning and may be used interchangeably throughout the description.

Embodiments of the disclosure will be described below in detail with reference to the accompanying drawings.

The disclosure relates to a robotic surgical system for minimally invasive surgery. The robotic surgical system will generally involve the use of multiple robotic arms. One or more of the robotic arms will often support a surgical tool which may be articulated (such as jaws, scissors, graspers, needle holders, micro dissectors, staple appliers, tackers, suction/irrigation tools, clip appliers, or the like) or non-articulated (such as cutting blades, cautery probes, irrigators, catheters, suction orifices, or the like). One or more of the robotic arms will often be used to support one or more surgical image capture devices such as an endoscope (which may be any of the variety of structures such as a laparoscope, an arthroscope, a hysteroscope, or the like), or optionally, some other imaging modality (such as ultrasound, fluoroscopy, magnetic resonance imaging, or the like).

The present disclosure is directed to robotic surgical systems, devices, methods, and computer-readable media that mitigate safety risks stemming from surgeon distraction from engagement with robotic surgical systems during surgical robotic procedures. More particularly, the present disclosure relates to systems and methods for identifying disengagement of a user using the robotic surgical system and causing the robotic surgical system to operate in one or more safe modes when the user is disengaged, thereby mitigating the risk that the user unintentionally injures the patient or otherwise compromises the surgical procedure by actuating the robotic surgical system while distracted. The systems and methods described herein provide various techniques for tracking a user position relative to a display of a surgeon console and, based on the tracked user position, determining whether the user is disengaged from a surgeon console, even for open-console architectures. If the user is disengaged from the surgeon console, the robotic surgical system is operated in one or more safe modes. Utilizing the technologies, techniques, and embodiments described herein, users are provided with a safer operating environment in which to perform robotic surgeries, and patients are afforded a safer environment in which to receive surgical treatment via robotic surgical systems.

Embodiments of the present disclosure are now described in detail with reference to the drawings in which like reference numerals designate identical or corresponding elements in each of the several views. As used herein, the terms “user” and “clinician” refer to a doctor, a surgeon, a nurse, technician, medical assistant, or similar support personnel or any other person that may use the robotic surgical systems described herein. Additionally, in the drawings and in the description that follows, terms such as front, rear, upper, lower, top, bottom, and similar directional terms are used simply for convenience of description and are not intended to limit the disclosure. In the following description, well-known functions or constructions are not described in detail to avoid obscuring the present disclosure in unnecessary detail.

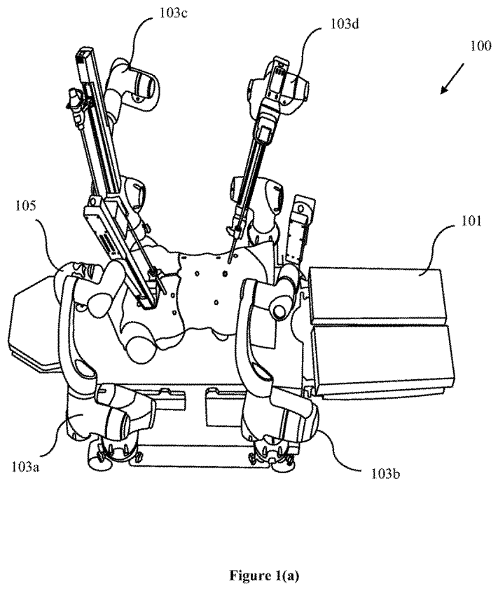

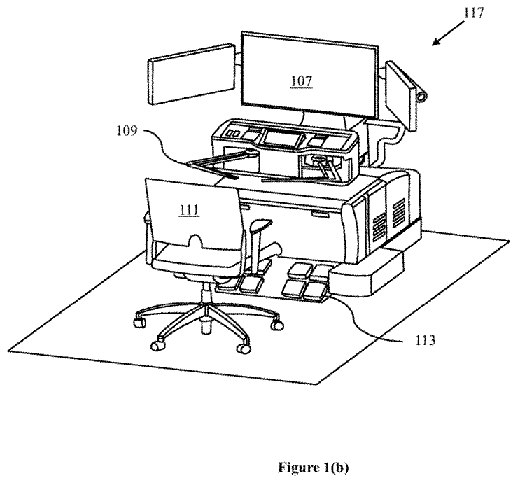

FIG. 1(a) illustrates a schematic diagram of multiple robotic arms of a robotic surgical system in accordance with an embodiment of the disclosure. Specifically, FIG. 1 illustrates the robotic surgical system (100) having four robotic arms (103a), (103b), (103c), (103d) mounted around a patient cart (101). The four-robotic arms (103a), (103b), (103c), (103d) as depicted in FIG. 1 is for illustration purpose and the number of robotics arms may vary depending upon the type of surgery or the robotic surgical system. The four robotic arms (103a), (103b), (103c), (103d) are mounted along the patient cart (101) and may be arranged in different manner but not limited to the robotic arms (103a), (103b), (103c), (103d) mounted on the patient cart (101) or the robotic arms (103a), (103b), (103c), (103d) separately mounted on a movable means or the robotic arms (103a), (103b), (103c), (103d) mechanically and/ or operationally connected with each other or the robotic arms (103a), (103b), (103c), (103d) connected to a central body (105) such that the robotic arms (103a), (103b), (103c), (103d) branch out of the central body (105).

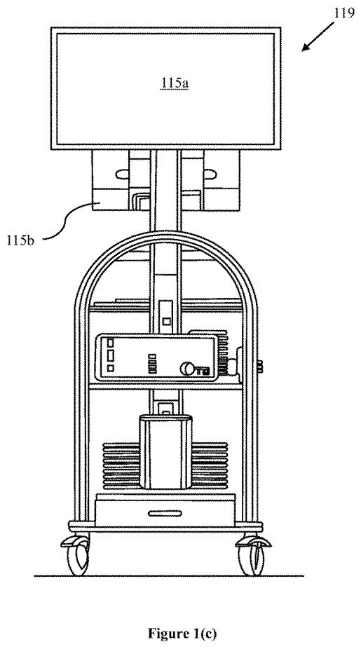

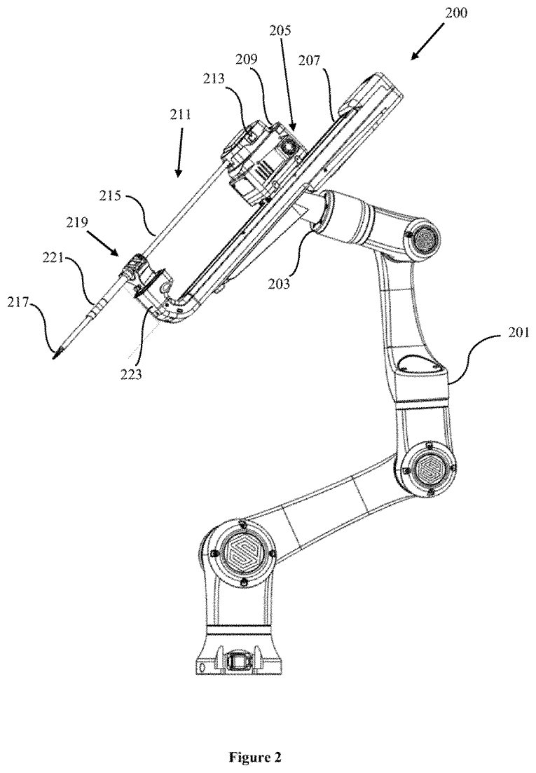

FIG. 1(b) illustrates a schematic diagram of a surgeon console of the robotic surgical system in accordance with an embodiment of the disclosure. The surgeon console (117) aids the surgeon to remotely operate the patient lying on the patient cart (101) by controlling the robotic arms (103a), (103b), (103c), (103d) inside the body of the patient. The surgeon console (117) is configured to control the movement of surgical instruments (as shown in FIG. 2(a)) while the instruments are inside the patient. The surgeon console (117) may comprise of at least an adjustable viewing means (107) but not limited to 2D/3D monitors, wearable viewing means (not shown) and in combination thereof. The surgeon console (117) may be equipped with multiple displays which would not only show 3D high definition (HD) endoscopic view of a surgical site at the patient cart (101) but may also shows additional information from various medical equipment's which surgeon may use during the robotic surgery. Further, the viewing means (107) may provide various modes of the robotic surgical system (100) but not limited to identification and number of robotic arms attached, current tool type attached, current tool tip position, collision information along with medical data like ECG, ultrasound display, fluoroscopic images, CT, MRI information. The surgeon console (117) may further comprise of mechanism for controlling the robotics arms but not limited to one or more hand controllers (109), one or more foot controllers (113), a clutch mechanism (not shown), and in combination thereof. The hand controllers (109) at the surgeon console (117) are required to seamlessly capture and transfer complex actions performed by surgeon giving the perception that the surgeon is directly articulating the surgical tools. The different controllers may require for different purpose during the surgery. In some embodiments, the hand controllers (109) may be one or more manually-operated input devices, such as a joystick, exoskeletal glove, a powered and gravity- compensated manipulator, or the like. These hand controllers (109) control teleoperated motors which, in turn, control the movement of the surgical instruments attached to the robotic arms. The surgeon may sit on a resting apparatus such as a chair (111), as depicted in FIG. 1(b), while controlling the surgeon console (117). The chair (111) may be adjustable with means in height, elbow rest and the like according to the ease of the surgeon and also various control means may be provided on the chair (111). Further, the surgeon console (117) may be at a single location inside an operation theatre or may be distributed at any other location in the hospital provided connectivity to the robotics arms is maintained.

FIG. 1(c) illustrates a schematic diagram of a vision cart of the robotic surgical system in accordance with an embodiment of the disclosure. The vision cart (119) is configured to display the 2D and/or 3D view of the operation captured by an endoscope. The vision cart (119) may be adjusted at various angles and heights depending upon the ease of view. The vison cart (119) may have various functionality but not limited to providing touch screen display, preview/recording/playback provisions, various inputs/outputs means, 2D to 3D converters and the like. The vision cart (119) may include a vision system portion (115a), (115b) such as TV screens, which enables a spectator or other non-operating surgeons to view a surgical site from outside the patient's body. One of the robotics arms typically engage a surgical instrument that has a video-image-capture function (i.e., a camera instrument) for displaying the captured images on the vision cart (119). In some robotic surgical system configurations, the camera instrument includes optics that transfer the images from the distal end of the camera instrument to one or more imaging sensors (e.g., CCD or CMOS sensors) outside of the patient's body. Alternatively, the imaging sensor(s) may be positioned at the distal end of the camera instrument, and the signals produced by the sensor(s) may be transmitted along a lead or wirelessly for processing and display on the vision cart (119).

FIG. 2 illustrates a perspective view of a tool interface assembly mounted on a robotic arm in accordance with an embodiment of the disclosure. The tool interface assembly (200) is mounted on the robotic arm (201) of the robotic surgical system (100). The tool interface assembly (200) is the main component for performing the robotic surgery on a patient. The robotic arm (201) as shown in FIG. 2 is shown for the illustration purpose only and other robotic arms with different configurations, degree of freedom (DOF) and shapes may be used.

The tool interface assembly (200), as depicted by FIG. 2, comprises of an ATI (Arm and Tool Interface) connector (203) which facilitates the tool interface (200) to operationally connect with the robotic arm. Further, the tool interface (200) further comprises of an actuator assembly (205) mounted on a guiding mechanism and capable of linearly moving along the guiding mechanism. The guiding mechanism depicted in FIG. 2 is a guide rail (207). The movement of the actuator assembly (205) along the guide rail (207) is controlled by the surgeon with the help of controllers on the surgeon console (117) as shown in FIG. 1(b). A sterile adapter assembly (209) is releasably mounted on the actuator assembly (205) to separate a non-sterile part of the robotic arm from a sterile surgical tool assembly (211). A locking mechanism (not shown) is provided to releasably lock and unlock the sterile adapter assembly (209) with the actuator assembly (205). The sterile adapter assembly (209) detachably engages from the actuator assembly (205) which drives and controls the sterile surgical instrument in a sterile field. In another embodiment, the surgical tool assembly (211) also may be releasably lock/unlock or engages/disengages with the sterile adapter assembly (209) by means of a push button (213).

The surgical tool assembly (211) includes a shaft (215) and end effectors (217). The end effector (217) may comprises of a surgical instrument or may be configured to attach a surgical instrument. The surgical tool assembly (211) may also contain stored (e.g., on a semiconductor memory inside the instrument) information that may be permanent or may be updatable by the robotic surgical system (100).

A cannula gripper (219) is provided on the tool interface assembly (200) and is configured to grip a cannula (221) which receives the shaft (215) through an opening (not shown). The cannula (221) comprises of a hollow body which comprises of grooves (not shown) in an internal surface (not shown). The grooves provide a locking mechanism that fixes the cannula (221) to the shaft (215) at desired angle and precludes shifting, twisting or any axial movement of the shaft (215) once received by the cannula (221). The cannula gripper (219) is detachably attached to one end of the tool interface assembly (200) and comprises of flap like body which receives the cannula (221). Alternatively, the cannula gripper (219) may have a circular body for receiving the cannula (221) and comprises of grooves to grip the cannula (221) at a stationary position.

The cannula gripper (219) may be affixed to the body of the tool interface assembly (200) and may be configured to grip or secure the cannula (221) such that cannula (221) is stable while performing surgical operations. The cannula gripper (219) may be affixed to a mount (223) of the tool interface assembly (200) by way of receiving the cannula gripper (219) within a set of grooves of the mount (223).

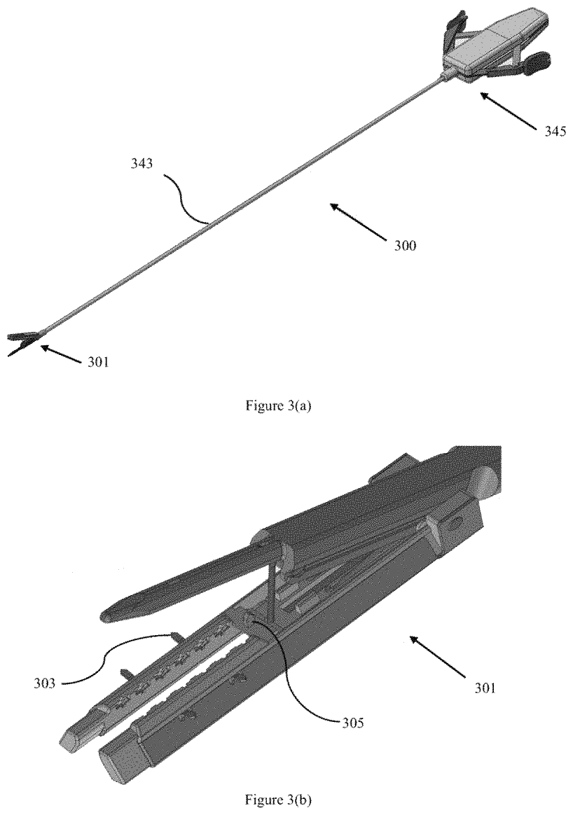

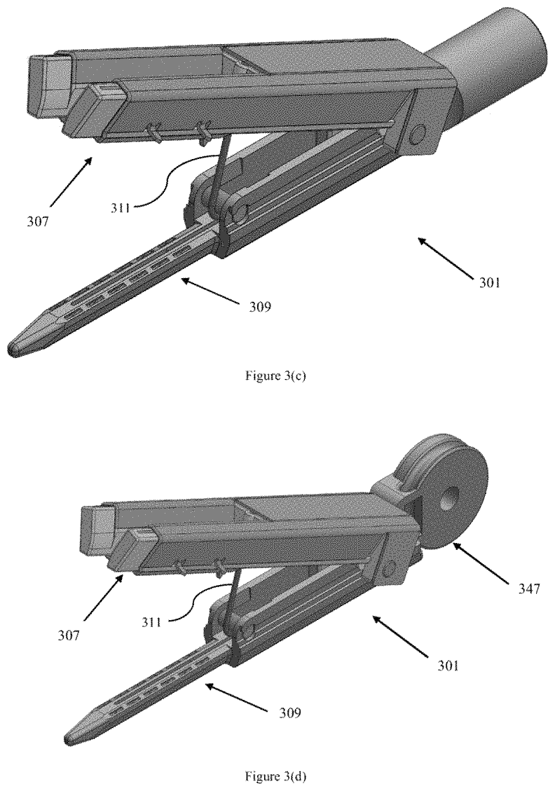

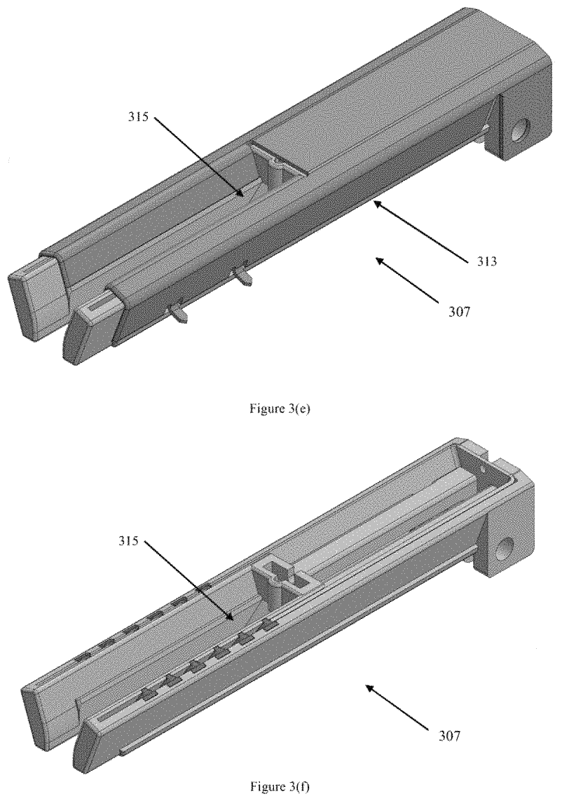

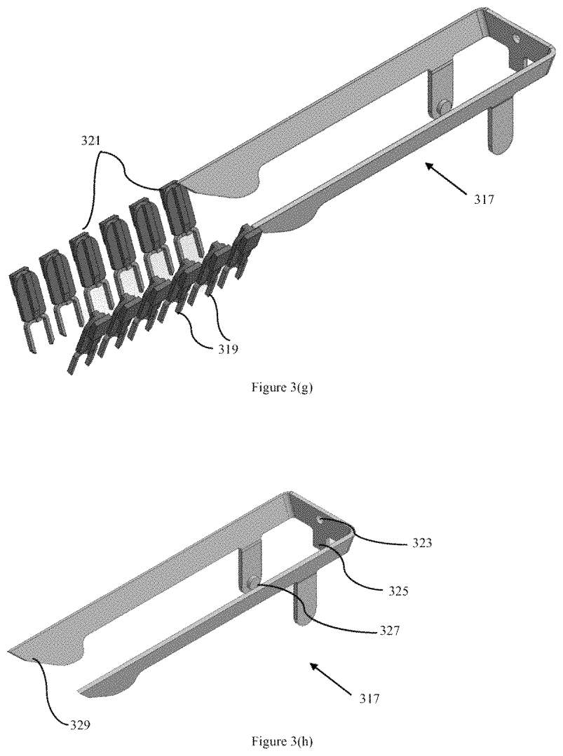

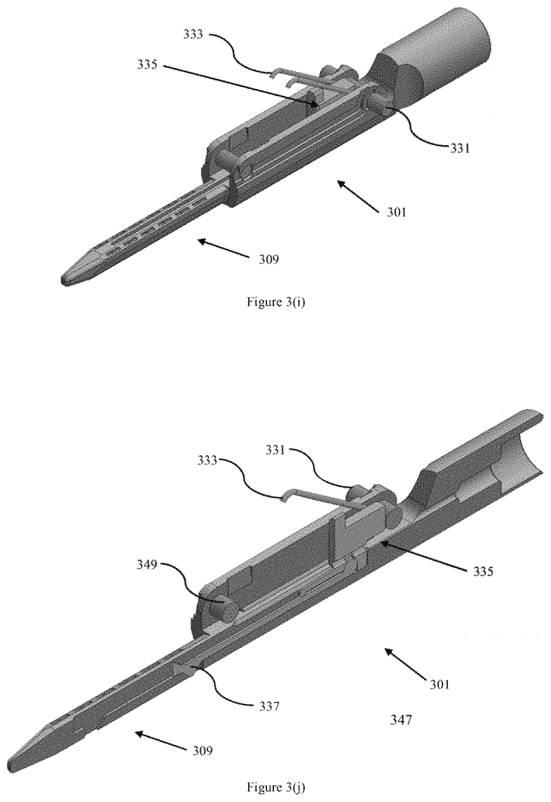

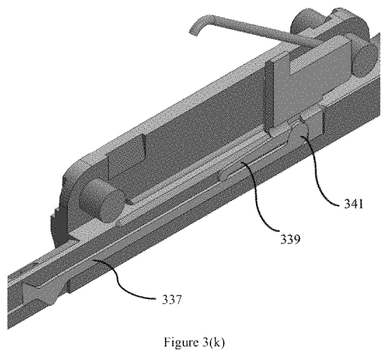

FIG. 3(a) illustrates a perspective view of an anastomosis device in accordance with an embodiment of the disclosure. The anastomosis device (300) is intended to create an end-to-side anastomosis between a graft vessel (not shown) and a target vessel (not shown). The anastomosis is created by the delivery of staples that connect the graft vessel to the target vessel and the creation of an incision to open a flow path between the graft vessel and the target vessel.

The anastomosis device (300) comprises of an end effector (301) that is designed to deliver staples to connect the vessels and create an incision to provide the required flow path as illustrated in FIG. 3(b). The anastomosis device (300) may comprise of a flexible shaft (343) connecting the end effector (301) to an interface (345) for connecting to a robot arm (not shown).

FIG. 3(c) and FIG. 3(d) illustrate the anastomosis device (300) in two configurations; manual and robotic. The manual configuration may also incorporate a handle for actuating the device (300) and a flexible shaft (not shown) that supports the Anastomosis end effector (301). The robotic configuration also incorporates a robot arm interface (not shown), a rigid shaft (not shown), a wrist (347), and the Anastomosis end effector (301).

In an embodiment, a method used for creating the anastomosis is fundamentally the same regardless of which device configuration is employed (manual or robotic). The first step is to prepare the graft and mount it on the end effector (301). The graft is prepared by creating a “hood.” The graft is positioned between the arms of the end effector (301) with the apex of the “hood” fitted onto a heel clip (305). The flaps of the “hood” are pressed onto a flap tines (303), as illustrated in FIG. 3(b).

The next step is to create a small incision in the target vessel. The anastomosis device (300) is positioned and an anvil (309) is inserted into the incision in the target vessel. When using the manual device configuration with the flexible shaft, positioning is facilitated by grasping the end effector (301) on the proximal hub using a grasper or other suitable instrument. When using the robotic configuration, positioning is facilitated by the ridged shaft rotation and the wrist (347) which provides pitch and yawl degrees of freedom.

After insertion, the anastomosis device (300) is actuated either manually by squeezing one or more handle actuation levers when using the manual configuration or remotely from the robot consul when using the robotic configuration. In either case, actuation of the device (300) initiates a sequence of actions at the end effector (301). First, the end effector (301) is clamped. This action clamps the anvil (309) (inside the target vessel) to a cartridge (307) (with the graft vessel mounted on it). Once the end effector (301) is fully clamped, the device (300) immediately proceeds with the deployment/forming of staples (319) and the creation of the incision which opens the required flow path between the graft vessel and the target vessel. When the actuation is complete, the end effector (301) has moved to a position to permit automatic unclamping when the actuation force is relaxed. Once the end effector (301) is unclamped, the anvil (309) can be withdrawn from the target vessel. It may be necessary to place a suture stitch to close the anvil entry hole.

FIG. 3(e) to FIG. 3(k) illustrates a perspective view of cartridge portions and anvil portions of the anastomosis device in accordance with an embodiment of the disclosure. Regardless of the instrument type (manual or robotic), the end effector (301) operation is the same. The end effector (301) consists of two major portions; the anvil (309) and the cartridge (307). The anvil (309) contains a knife (337) and knife block (335) for creating the incision in the target vessel that establishes the required flow path, as illustrated in FIG. 3(j). The cartridge (307) contains a body (315) and a metal cap (313), where the body (315) contains staples (319), staple drivers (321), and wedge (317) to actuate the drivers (321), as illustrated in FIG. 3(g). The end effector (301) has been designed to execute the required sequence of operation with the actuation of a single cable (311). This is a significant simplification over other anastomosis devices which have four or more cables that are required.

Individual staples (319) are used to connect the vessels. These staples (319) are arranged in bays molded into both arms of the cartridge (307). The staples (319) are formed against pockets in the anvil (309). The staples (319) are advanced by individual drivers (321)—one for each staple. The drivers (321) are sequentially actuated proximal to distal via a dual wedge (317). The dual wedge (317) has two legs—one leg for each arm of the cartridge (307)—with a cam (327) for moving the drivers (321) disposed on the end of each leg. The dual wedge (317) comprises of a hole (323), an actuation tab (325), a driver actuation tab (329), and the sequence cam (327).

The knife (337) is positioned within the anvil (309) of the end effector (301) for creating the incision in the target vessel. The initial position of the knife (337) is retracted for easy insertion of the anvil (309) into the target vessel. As the staples (319) are formed, the knife (337) advances on a ramped surface on the anvil (309) to lift the knife (337)—cutting the tissue. Just prior to the end of stroke, the knife (337) moves down a ramp back to the retracted position within the anvil (309) for ease of removal. The knife (337) is rotationally mounted on a knife block (335). The knife block (335) has a spring finger (339) to keep the knife (337) riding on the ramps. The knife block (335) is advanced when a tab on the wedge (317) engages it. Specifically, the wedge tab (325) is proximal of the knife block (335) initially. As the wedge (317) advances distally, the first staple on either side of the anastomosis is formed. Then the tab (325) of the wedge (317) engages the knife (337) so that the cut is initiated after the first staple (319) is formed. Similarly, as the wedge (317) continues forward, the knife (337) continues to cut the tissue just proximal of the next forming staple. Finally, the knife (337) is retracted on the ramp as the last staple is formed. The result is a cut that begins just distal of the first formed staple and stops just proximal of the last formed staple.

In order to activate the instrument with a single cable, the design takes advantage of the fact that clamping always precedes wedge (317) advancing. The single deployment cable (311) is attached to the wedge (317). The cable (311) is routed from the wedge (317) around a guide pin (331) and down to a second guide pin (349) on the anvil (309) and then out the proximal end of the end-effector (301). In order to ensure that the wedge (317) does not advance when the cable (311) is pulled until the anvil (309) and cartridge (307) are clamped, legs with cam posts are provided on both sides of the wedge (317). Initially, when the anvil (309) and cartridge (307) are unclamped (due to a leaf spring (333) that forces them open), the cam posts are in a track that only permits rotation of the wedge (317) as required for clamping. Once fully clamped the cam posts align with a new section of track that allows the wedge (317) to advance. Once the wedge (317) gets to the final distal position, the cam path opens up again allowing the wedge (317) (and cartridge (307)) to rotate back to the unclamped position.

The foregoing descriptions of exemplary embodiments of the present disclosure have been presented for purposes of illustration and description. They are not intended to be exhaustive or to limit the disclosure to the precise forms disclosed, and obviously many modifications and variations are possible in light of the above teaching. The exemplary embodiment was chosen and described in order to best explain the principles of the disclosure and its practical application, to thereby enable others skilled in the art to best utilize the disclosure and various embodiments with various modifications as are suited to the particular use contemplated. It is understood that various omissions, substitutions of equivalents are contemplated as circumstance may suggest or render expedient but is intended to cover the application or implementation without departing from the spirit or scope of the claims of the present disclosure.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

While specific language has been used to describe the disclosure, any limitations arising on account of the same are not intended. As would be apparent to a person in the art, various working modifications may be made to the apparatus in order to implement the inventive concept as taught herein.

We claim:

1. An anastomosis device comprising:
an interface (345);
a flexible shaft (343); and
an end effector (301), the flexible shaft (343) connecting the interface (345) and the end effector (301), the end effector (301) including an anvil (309) extending from the end effector (301) and a cartridge (307) pivotably attached to the end effector (301), the anvil (309) including a knife (337) and knife block (335), the cartridge (307) including a body (315) for receiving at least one tissue fastener.
